(19) United States
(12) Patent Application Publication
(10) Pub. No.: US 2004/0019567 A1
(43) Pub. Date: Jan. 29, 2004

(54) Electronic prescription ordering method, system, and program product

(75) Inventors: Michael J. Herceg, Johnson City, NY (US); Daniel L. Krissell, Kirkwood, NY (US); Thomas E. Murphy, JR., Vestal, NY (US); Francine M. Orzel, Endicott, NY (US); Paul F. Rieth, Apalachin, NY (US); Jeffrey S. Stevens, Endicott, NY (US)

Correspondence Address: HOFFMAN WARNICK & D'ALESSANDRO, LLC, 3 E-COMM SQUARE, ALBANY, NY, 12207

(73) Assignee: International Business Machines Corporation, Armonk, NY

(21) Appl. No.: 10/202,424

(22) Filed: Jul. 23, 2002

(51) Int. Cl. G06F 17/60

(52) U.S. Cl. 705/64; 705/2

(57) ABSTRACT

An electronic prescription ordering method, system, and program product. The invention provides a method, system, and program product for electronically processing a prescription request that allow a user to request and compare prescription information for a group of pharmaceutical suppliers. The user can then electronically place a prescription order with a pharmaceutical supplier based on the prescription information that was returned.

### BACKGROUND OF THE INVENTION

1. Technical Field

The current invention relates to ordering prescriptions. More particularly, the invention provides a system, method, and program product for electronically ordering a prescription from a pharmaceutical supplier.

2. Background Art

Prescriptions are becoming an ever-increasing part of the lives of many individuals. Currently, individuals visit a physician who may determine that a prescription is required. The physician then writes out the prescription on a prescription pad and gives the prescription to the patient. The patient must then take the prescription to a pharmaceutical supplier (i.e., pharmacy) that fills the prescription. This process contains many problems, annoyances, and expenses for the pharmacist, patient, and physician. For example, a physician's handwriting is not always legible and patients are frequently unaware of the precise name of the drug being prescribed. As a result, a pharmacist may be required to interpret the physician's handwriting. Misinterpretation of the handwriting can result in misfilled prescriptions that may have serious consequences. Further, handwritten prescriptions are highly susceptible to tampering and forgery.

Additionally, from a consumer's perspective, there are many difficulties associated with purchasing the prescribed medication. For example, once the prescription is presented to the pharmacist, the individual must wait for it to be filled. Depending on how busy the pharmacist is at the time, this can take anywhere from a few minutes to hours. When an individual is feeling ill, this wait can be extremely uncomfortable. A pharmacy also may be temporarily out of a particular drug. The individual generally will not learn this until the prescription is presented at the pharmacy. In this case, the individual will need to go to another pharmacy, thereby incurring additional travel time, or wait for the pharmacy to resupply, requiring an additional trip. Further, an individual may find out that the pharmacy does not accept his/her insurance or method of payment, thereby necessitating a trip to another pharmacy.

It is also difficult for individuals to shop around for the best price in filling a prescription. This can result in higher prices being paid by the individual or insurer, and higher insurance premiums. Individuals may also be hesitant to shop around at various pharmacies for fear that a dangerous drug interaction will not be detected, especially when more than one physician is being consulted and multiple prescriptions are being taken.

Further, some individuals may prefer to have a prescription delivered to their house. In this instance, the need to personally place the order at a pharmacy limits the usefulness of this option. Additionally, many regional laws prohibit fulfilling certain prescriptions through the mail.

As a result, there exists a need for a method, system, and program product for electronically ordering a prescription. The method, system, and program product can allow for an individual and/or physician (i.e., a prescriber) to request that a group of pharmacies provide prescription information for a particular prescription. The individual and/or physician can then compare the various responses, select one of the pharmacies based on the prescription information and place an order electronically.

### SUMMARY OF THE INVENTION

The current invention provides a method, system, and program product for electronically processing a request for a prescription. The invention allows several pharmaceutical suppliers to be compared using various information on the prescription and other criteria. A desired pharmaceutical supplier can then be selected and an order placed. The invention can allow for automated checking for drug interactions and inventories of the various pharmaceutical suppliers. Further, an individual can have a prescription delivered, thereby alleviating the requirement to go to a particular pharmaceutical supplier in person.

A first aspect of the invention provides a method for electronically ordering a prescription, comprising: receiving a prescription request from a prescriber; processing the prescription request according to at least one selection criterion to identify at least one target pharmaceutical supplier from a group of pharmaceutical suppliers; forwarding the prescription request to the at least one target pharmaceutical supplier; receiving responses to the prescription request from the at least one target pharmaceutical supplier; and forwarding the responses to the prescriber.

A second aspect of the invention provides a method for electronically ordering a prescription, comprising: receiving a prescription request from a prescriber; processing the prescription request according to at least one selection criterion specified in the prescription request to identify target pharmaceutical suppliers from a group of pharmaceutical suppliers; forwarding the prescription request to the target pharmaceutical suppliers; receiving responses to the prescription request from the target pharmaceutical suppliers; forwarding the responses to the prescriber; receiving an order request from the prescriber for one of the target pharmaceutical suppliers; validating the order request; and forwarding the order request to the one of the target pharmaceutical suppliers.

A third aspect of the invention provides a system for electronically ordering a prescription, comprising: a prescriber system for receiving a prescription request from a prescriber and for forwarding responses to the prescriber; a processing system for processing the prescription request according to at least one selection criterion to identify at least one target pharmaceutical supplier from a group of pharmaceutical suppliers; and a supplier system for forwarding the prescription request to the at least one target pharmaceutical supplier and for receiving responses to the prescription request from the at least one target pharmaceutical supplier.

A fourth aspect of the invention provides a system for electronically ordering a prescription, comprising: a prescriber system for receiving a prescription request and an order request from a prescriber and for forwarding responses to the prescriber; a processing system for processing the prescription request according to at least one selection criterion specified in the prescription request to identify target pharmaceutical suppliers from a group of pharmaceutical suppliers; a supplier system for forwarding the prescription request to the target pharmaceutical suppliers, receiving responses to the prescription request from the target pharmaceutical suppliers, and forwarding the order request to one of the target pharmaceutical suppliers; and a validation system for validating the order request.

A fifth aspect of the invention provides a program product stored on a recordable medium for electronically ordering a prescription, which when executed, comprises: program code for receiving a prescription request from a prescriber; program code for processing the prescription request according to at least one selection criterion to identify at least one target pharmaceutical supplier from a group of pharmaceutical suppliers; program code for forwarding the prescription request to the at least one target pharmaceutical supplier; program code for receiving responses to the prescription request from the at least one target pharmaceutical supplier; and program code for forwarding the responses to the prescriber.

A sixth aspect of the invention provides a program product stored on a recordable medium for electronically ordering a prescription, which when executed, comprises: program code for receiving a prescription request from a prescriber; program code for processing the prescription request according to at least one selection criterion specified in the prescription request to identify target pharmaceutical suppliers from a group of pharmaceutical suppliers; program code for forwarding the prescription request to the target pharmaceutical suppliers; program code for receiving responses to the prescription request from the target pharmaceutical suppliers; program code for forwarding the responses to the prescriber; program code for receiving an order request from the prescriber for one of the target pharmaceutical suppliers; program code for validating the order request; and program code for forwarding the order request to the one of the target pharmaceutical suppliers.

The exemplary aspects of the present invention are designed to solve the problems herein described and other problems not discussed, which are discoverable by a skilled artisan.

### BRIEF DESCRIPTION OF THE DRAWINGS

These and other features of this invention will be more readily understood from the following detailed description of the various aspects of the invention taken in conjunction with the accompanying drawings in which:

FIG. 1 is a schematic overview of a system according to one aspect of the invention;

FIG. 2 is a more detailed diagram of the central pharmaceutical computer of FIG. 1;

FIG. 3 is a method of processing a prescription request according to one aspect of the invention; and

FIG. 4 is a method of processing an order according to another aspect of the invention.

It is noted that the drawings of the invention are not to scale. The drawings are intended to depict only typical aspects of the invention, and therefore should not be considered as limiting the scope of the invention. In the drawings, like numbering represents like elements between the drawings.

### DETAILED DESCRIPTION OF THE INVENTION

The current invention provides a method, system, and program product for electronically ordering a prescription. The invention can provide a physician and/or patient with the ability to select a pharmaceutical supplier based on one or more criteria. Further functionality can be provided, such as determining whether a dangerous drug interaction exists with other medications that a patient may be taking.

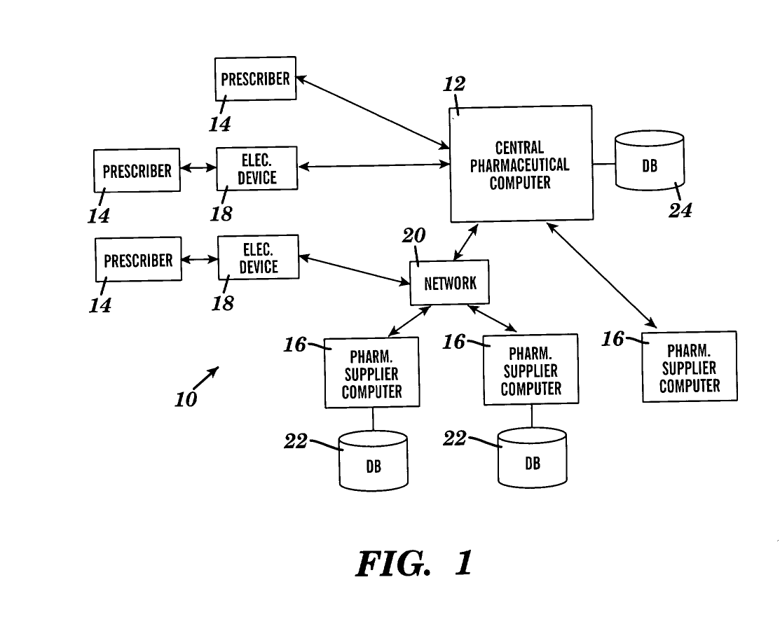

Turning to FIG. 1, a typical embodiment of the invention is shown. System 10 is shown including a central pharmaceutical computer 12 that provides processing and communications between one or more prescribers 14 and one or more pharmaceutical supplier computers 16. Prescribers 14 can be any individual/entity that writes prescriptions including a primary care physician, a specialty physician, a veterinarian, etc. Moreover, prescriber 14 can also be an individual seeking to fill a prescription (i.e., patient, parent of patient, owner of pet, etc.). For example, while system 10 can generally be used to allow a physician and patient to bypass writing out a prescription and going to a pharmaceutical supplier with the written prescription, a patient can use system 10 to shop around at various pharmaceutical suppliers and order a prescription that is subsequently verified by presenting a hand written prescription when the individual picks the medication up. Pharmaceutical supplier computers 16 are typically computers for various pharmaceutical suppliers. A pharmaceutical supplier can comprise a drug store, a superstore that includes a pharmacy, a mail order pharmacy, a chain of pharmacies/superstores, etc.

As shown, prescribers 14 can communicate with central pharmaceutical computer 12 directly, using an electronic device 18, and/or using an electronic device 18 connected to a network 20. Electronic device 18 can comprise any type of device capable of accepting input, providing output, and communicating with another device. For example, electronic device 18 can be a mobile phone, a handheld computer, a personal digital assistant, a portable (e.g., laptop) computer, a desktop computer, a mainframe computer, etc. As shown electronic device 18 can be used to communicate directly with central pharmaceutical computer 12 (e.g., a touchscreen device) or to connect to network 20, which provides a communications path to central pharmaceutical computer 12 (e.g., a laptop computer connected to the Internet). In addition, communications between electronic device 18, central pharmaceutical computer 12, and/or network 20 can be implemented using any method or combination of methods, including, wireless, satellite, ethernet, fiber optic, serial, parallel, etc.

Similarly, pharmaceutical supplier computers 16 can communicate with central computer 12 directly and/or over network 20. Network 20 can comprise any type of network, including, for example, a local area network (LAN), wide area network (WAN), a virtual private network (VPN), other private networks, a global network, the Internet, etc. While prescribers 14 and pharmaceutical supplier computers 16 are shown communicating with central pharmaceutical computer 12 over the same network 20, it is understood that different and/or multiple networks 20 can be used by prescribers 14 and pharmaceutical supplier computers 16.

Central pharmaceutical computer 12 is shown having a database 24. Further, some of pharmaceutical supplier computers 16 are shown having a database 22. Databases 22, 24 can be used in storing and retrieving data necessary for implementing the invention as further described below.

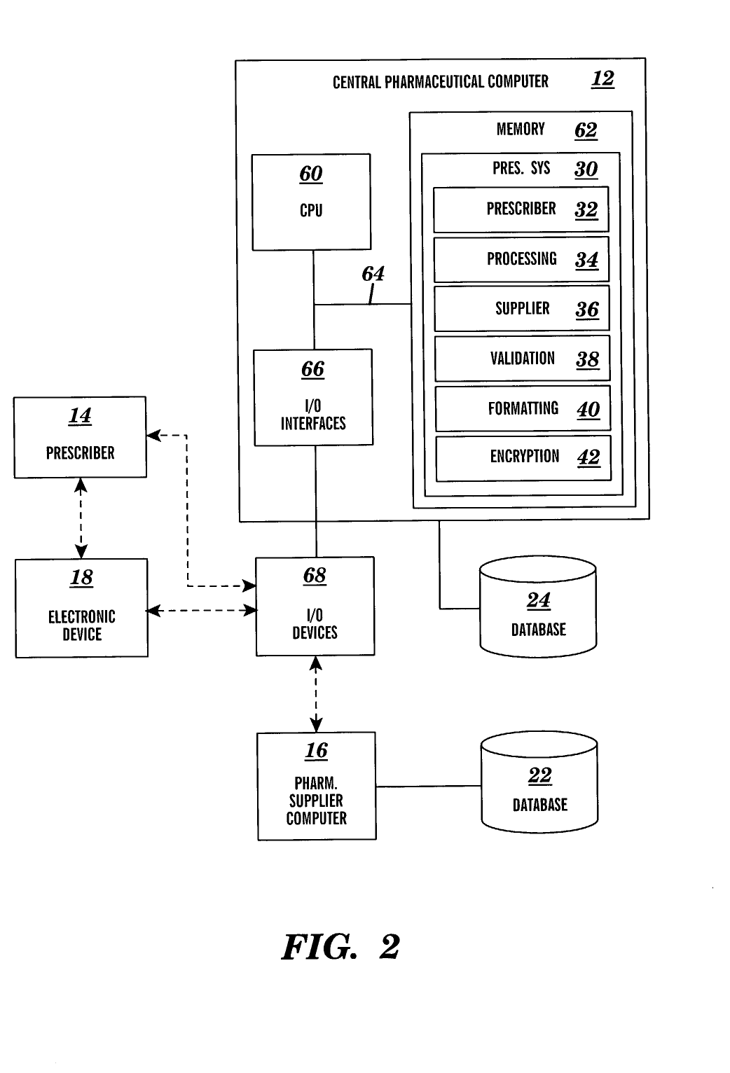

FIG. 2 provides more detailed diagram of central pharmaceutical computer 12 of FIG. 1. As shown, central pharmaceutical computer 12 generally comprises central processing unit (CPU) 60, memory 62, bus 64, input/output (I/O) interfaces 66, I/O devices 68 and database 24. CPU 60 may comprise a single processing unit, multiple processing units capable of parallel operation, or be distributed across one or more processing units in one or more locations, e.g., on a client and server. Central pharmaceutical computer 12 can comprise an advanced mid-range multiprocessor-based server utilizing standard operating system software, which is designed to drive the operation of the particular hardware and which is compatible with other system components and I/O controllers. Memory 62 may comprise any known type of data storage and/or transmission media, including magnetic media, optical media, random access memory (RAM), read-only memory (ROM), a data cache, a data object, etc. Moreover, similar to CPU 60, memory 62 may reside at a single physical location, comprising one or more types of data storage, or be distributed across a plurality of physical systems in various forms.

I/O interfaces 66 may comprise any system for exchanging information with one or more I/O devices 68. I/O devices 68 may comprise any known type of input/output device, including an I/O port (serial, parallel, ethernet, etc.), a universal serial bus (USB) controller, a network system, modem, speakers, a monitor (cathode-ray tube (CRT), liquid-crystal display (LCD), etc.), hand-held device, keyboard, mouse, voice recognition system, speech output system, scanner, printer, facsimile, pager, storage devices, etc. Bus 64 provides a communication link between each of the components in central pharmaceutical computer 12 and likewise may comprise any known type of transmission link, including electrical, optical, wireless, etc. In addition, although not shown, additional components, such as cache memory, communication systems, system software, etc., may be incorporated into central pharmaceutical computer 12.

Database 24 may provide storage for information necessary to carry out the present invention. Such information could include, among other things, prescriber information, patient records, pharmaceutical supplier information, drug information, etc. As such, database 24 may include one or more storage devices, such as a magnetic disk drive or an optical disk drive. In another embodiment, database 24 includes data distributed across, for example, a LAN, WAN or a storage area network (SAN) (not shown). Database 24 may also be configured in such a way that one of ordinary skill in the art may interpret it to include one or more storage devices. It is understood that although not shown, electronic device 18 and pharmaceutical supplier computer 16 typically contain components (e.g., CPU, memory, etc.) similar to central pharmaceutical computer 12. Such components have not been separately depicted and described for brevity purposes.

As discussed previously, communication between central pharmaceutical computer 12, electronic device 18 and pharmaceutical supplier computer 16 can be provided through any known means. For example, central pharmaceutical computer 12, electronic device 18 and pharmaceutical supplier computer 16 can be connected via direct hardwired connections (e.g., cable) through their respective I/O devices 68 (e.g., serial port), or via addressable connections (e.g., remotely) in a client-server environment. In the case of the latter, the server and client may be connected via the Internet, WAN, LAN, or other private networks. The server and client may utilize conventional network connectivity, such as Token Ring, Ethernet, or other conventional communications standards. Where the client communicates with the server via the Internet, connectivity could be provided by conventional TCP/IP sockets-based protocol. In this instance, the client could utilize an Internet service provider to establish connectivity to the server.

Prescriber 14 and pharmaceutical supplier computer 16 can interact with central pharmaceutical computer 12 to provide, modify, and/or retrieve information. To this extent, prescriber 14 may interact with central pharmaceutical computer 12 by interacting directly with I/O devices 68, and/or by interacting with electronic device 18 in communication with one or more I/O devices 68 in a network environment. For example, central pharmaceutical computer 12 can be connected to the world wide web by using one or more I/O devices 68. Prescriber 14 can subsequently access the world wide web using electronic device 18 and interact with central pharmaceutical computer 12.

Prescription system 30 is shown stored in memory 62 (as a program product) and includes prescriber system 32, processing system 34, supplier system 36, validation system 38, formatting system 40, and encryption system 42. While the systems are all shown in memory 62, it is understood that some or all of these systems can be stored in memory on electronic device 18 and/or pharmaceutical supplier computer 16. Further, portions of one or more of the systems can be duplicated and/or stored on electronic device 18 and/or pharmaceutical supplier computer 16.

In general, prescriber 14 sends a prescription request to central pharmaceutical computer 12 which can forward the prescription request to one or more pharmaceutical supplier computers 16. Some or all of the pharmaceutical supplier computers 16 can generate responses to the prescription request. Central pharmaceutical computer 12 then forwards these responses to prescriber 14.

Prescriber system 32 receives all prescription requests from prescriber 14. A prescription request may be encrypted, in which case encryption system 42 can be used to decrypt the prescription request. Once an unencrypted prescription request is available, validation system 38 can be used to validate the prescription request. This may include ensuring that a valid physician submitted the prescription request based on a key, a unique identifier, electronic signature, etc.

Valid prescription requests are then processed using processing system 34 to identify target pharmaceutical suppliers. Specifically, processing system 34 processes the prescription request according to at least one selection criterion to identify target pharmaceutical suppliers from a group of pharmaceutical suppliers. Selection criteria may be included in a particular prescription request and/or generated based on known attributes of prescriber 14 and/or patient (e.g., physician's office location, patient's home address). Selection criteria can include, for example, a prescription price or price range, a pharmaceutical supplier location, a prescription availability, an acceptance of an insurer, an availability of local pharmaceutical supplier data, an acceptance of a method of payment, a previous use, a delivery option, an amount of time to fill, etc. Any combination of selection criteria can be used for a particular prescription request, and some criteria can be selected by prescriber 14 while other criteria can be generated by processing system 34. To this extent, “target pharmaceutical suppliers” are the pharmaceutical suppliers that meet the requirements of the request.

Once the target pharmaceutical suppliers have been identified, the prescription request can be forwarded to each target pharmaceutical supplier computer(s) 16 using supplier system 36. Communication to the various target pharmaceutical suppliers can be held in series or parallel. When non-homogenous data formats are used by the target pharmaceutical suppliers, formatting system 40 can format the prescription request for each of the pharmaceutical supplier computers. Should encryption be desired, the reformatted prescription request may be encrypted using encryption system 42.

For some target pharmaceutical suppliers, central pharmaceutical computer 12 may access database 24, which has the required data to generate a response to the prescription request. For example, a pharmaceutical supplier can implement a system in which pharmaceutical supplier computer 16 communicates availability and pricing information of its various drugs to central pharmaceutical computer 12 on a daily basis. Central pharmaceutical computer 12 can store and update this information in database 24. Other more static information regarding the pharmaceutical supplier can also be stored in database 24. When one of these pharmaceutical suppliers is a target pharmaceutical supplier, processing system 34 can access the necessary data in database 24 and generate a response for the pharmaceutical supplier (i.e., without forwarding the request to the pharmaceutical supplier).

When a prescription request is forwarded to pharmaceutical supplier computer 16, it can decrypt, validate, and process the prescription request in a similar manner as central pharmaceutical computer 12 to generate a response. Database 22 can be used to store the necessary data that is incorporated into the response. A response can include, for example, a price for the prescription, a time estimate as to when the prescription will be filled, alternate locations that can fill the prescription, etc. Once a response is generated, pharmaceutical supplier computer 16 can encrypt it, if desired, and forward the response to central pharmaceutical computer 12. Supplier system 36 can be used to receive the response from the target pharmaceutical supplier computers 16.

Once a response is received by supplier system 36, it may be decrypted by encryption system 42, validated by validation system 38 and/or formatted by formatting system 40 in order to be forwarded to prescriber 14. Additionally, processing system 34 may incorporate additional data in the response that was available locally (i.e., delivery option, valid method of payments, etc.) and did not require forwarding to pharmaceutical supplier computer 16. Once complete, encryption system 42 can re-encrypt the response and prescriber system 32 can forward the response to the prescriber.

Since communications between prescriber 14, central pharmaceutical computer 12, and pharmaceutical supplier computers 16 may be implemented over a WAN, the Internet, etc., some time may elapse before a response is received. Processing for forwarding the prescription request to other target pharmaceutical suppliers can continue during this time. A “time out” can be incorporated after which it is assumed that a pharmaceutical supplier computer is not going to supply a response to a prescription request. In this case, once the period expires, all responses that have been received can be forwarded to prescriber 14 as a single packet. Alternatively, each response can be forwarded when complete, and the prescriber can merely determine how long he/she desires to wait before selecting a desired pharmaceutical supplier to fill the prescription.

Once a desired pharmaceutical supplier is selected, prescriber 14 can send an order to central pharmaceutical computer 12. It is understood, however, that prescriber 14 may not desire to place an order based on the responses that are received from the target pharmaceutical suppliers. In this case, a second prescription request using different criteria can be generated, or a traditional prescription can be written out and filled outside the system. After reviewing the responses, prescriber 14 may select a pharmaceutical supplier to fill the prescription. Prescriber 14 can forward an order for the prescription to central pharmaceutical computer 12 (which is received by prescriber system 32) that can then forward the order to a pharmaceutical supplier computer 16 via supplier system 36. Pharmaceutical supplier computer 16 can forward a confirmation that the order was received and will be filled to central pharmaceutical computer 12, which then forwards the confirmation to prescriber 14.

When an order is received, as with a prescription request, the order can be decrypted using encryption system 42, validated using validation system 38, and/or formatted using formatting system 40 for the pharmaceutical supplier that was selected to fill the prescription. Further, the order can be re-encrypted by encryption system 42 and forwarded to pharmaceutical supplier computer 16 by supplier system 36. When local data is stored for the pharmaceutical supplier (i.e., in database 24), it can be updated to reflect the order. For example, an amount of the prescribed drug that is available can be deducted by the prescribed amount. Pharmaceutical supplier computer 16 can then process the order in a similar fashion, update its database 22, and inform the appropriate individual (i.e., pharmacist) to fill the prescription. If desired, pharmaceutical supplier computer 16 can generate a confirmation that the order was received and will be filled by the selected pharmaceutical supplier. The confirmation can include data such as the time that the prescription will be available, the time that it will be delivered to a particular location (i.e., patient's residence), a confirmation number to be presented when the prescription is to be picked up, etc.

Additional functionality can also be included in the central pharmaceutical computer 12. For example, central pharmaceutical computer 12 can maintain data on all patients for a particular insurer. In this case, a patient may see numerous specialists for various disorders. When a specialist desires to prescribe a medication, he/she can use the system herein described. Depending on the amount of information in the prescription request and the order, the medication can be screened for any dangerous interactions for other medications that the patient is currently taking. For patient confidentiality, a prescription request may only inquire about the availability and price of a prescribed drug. In this case, central pharmaceutical computer 12 does not have sufficient information to determine whether a dangerous interaction will occur. However, when an order is received, this information would be available and can be checked.

To maximize the number of prescribers and pharmaceutical suppliers that can use the invention, and to minimize the amount of customization required to implement the invention, the system can be implemented in a platform independent manner. For example, prescription system 30 can be at least partially written in Extensible Markup Language (XML) and use Document Type Definitions (DTD) as are commonly used in many business-to-business Internet-based solutions. In this case, prescriber 14 may only require access to a device that has a web browser.

Below is a sample of what a prescription request written in XML might look like:

<?xml version=“1.0”?>
<!DOCTYPE RxXML SYSTEM “RxRequest.dtd”>
<RxXML timestamp=“2000-12-02T11:29:45-05:00” version=“1.1”>

<Header>

<SenderInfo>

<Name>East Side Health Offices</Name>
<physician>John M Dee</physician>
<Phone>555-555-5555</Phone>
<Address>

<Street>12 main st.</Street>
<City>Endicott</City>
<State>NY</State>
<Zip>13760</Zip>

</Address>
<RxIdInfo>

<!-- Some unique physician id and associated
password -->

</RxIdInfo>

</SenderInfo>
<PharmacyInfo>

<Name>CVC Pharmacy</Name>
<Phone>555-555-6666</Phone>
<Address>

<Street>124 main st.</Street>
<City>Endicott</City>
<State>NY</State>
<Zip>13760</Zip>

</Address>
<RxInfo>

<!-- Some unique pharmacy id and infomation -->

</RxInfo>

</PharmacyInfo>

</Header>
<Body>

<NewPrescriptionRequest>

<Patient>

<Name>Joe Doe</Name>
<Age>35</Age>
<Phone>666-555-5656</Phone>
<Address>

<Street>43 East ave.</Street>
<City>Endicott</City>
<State>NY</State>
<Zip>13760</Zip>

</Address>

</Patient>
<Insuranceinformation>
<Insurer name=“New York Insurance Co.”>

<Phone>555-555-1212</Phone>
<Address>

<Street>1 braod st.</Street>
<City>New York</City>
<State>NY</State>
<Zip>10010</Zip>

</Address>

</Insurer>
<Subscriber name=“Joe Doe”/>
<ID>123456789</ID>
<Group>W902</Group>
<Plan name=“A” />

</InsuranceInformation>
<Prescription>

<Name>Zantac</Name>
<Strength>15 mg/ml</Strength>
<Dosage>3 ml, 2X daily</Dosage>
<Quantity>30</Quantity>
<Refills>0</Refills>
<DispenseAsWritten>Yes</DispenseAsWritte>
<SpecialInstructions>Some special instructions for

pharmacist.........</SpecialInstructions>

<SpecialInstructions>Another special

instruction.</SpecialInstructions>

<SpecialInstructions>One

more.</SpecialInstructions>

</Prescription>
<Prescription>

<Name>Prozac</Name>
<Strength>150 mg</Strength>
<Dosage>1 pill, 3X daily</Dosage>
<Quantity>60</Quantity>
<Refills>3</Refills>
<DispenseAsWritten>No</DispenseAsWritten>

</Prescription>

</NewPrescriptionRequest>

</Body>

</RxXML>

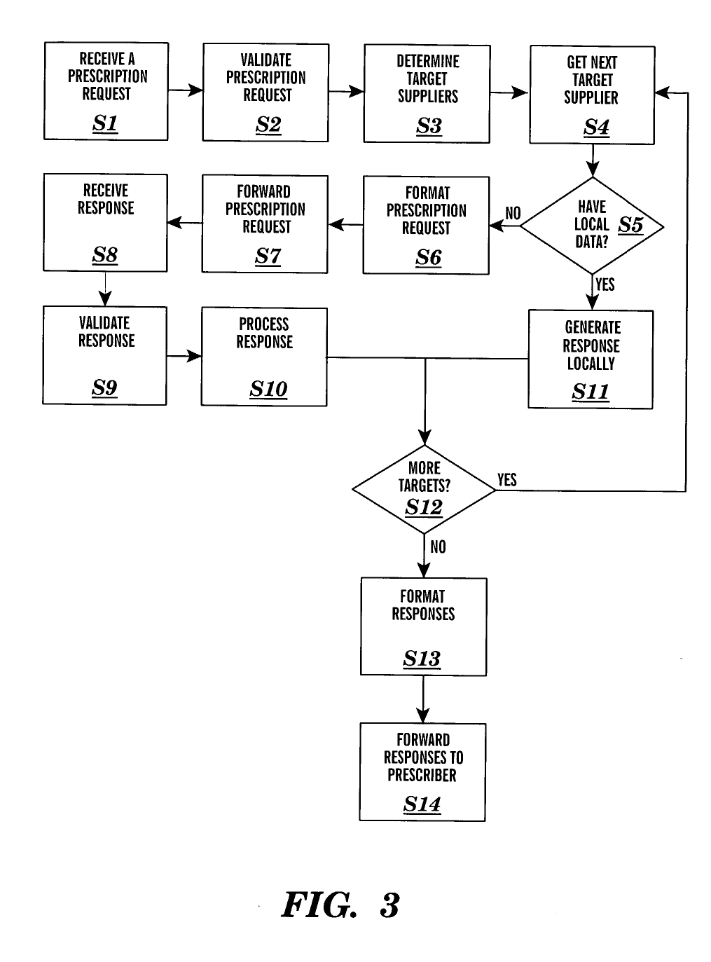

FIG. 3 depicts an exemplary method of processing a prescription request. Initially, a prescription request is received in step S1. The prescription request is then validated in step S2. Target pharmaceutical supplier(s) are identified in step S3 based on the prescription request, selection criterion, and/or other data. Specifically, based on the requested prescription and any other requirements (e.g., price, location, etc.), specific “target” pharmaceutical suppliers will be identified. Once the target pharmaceutical suppliers are determined, each target pharmaceutical supplier is then processed starting in step S4.

When processing a target pharmaceutical supplier, it is initially determined in step S5 whether there is local data (e.g., price and availability information) for a particular target pharmaceutical supplier so that a response can be generated without forwarding the prescription request. When no local data is present, the prescription request is formatted appropriately in step S6 and forwarded to the target pharmaceutical supplier in step S7. The target pharmaceutical supplier then generates and forwards a response to the prescription request that is received in step S8. A received response is then validated in step S9 and processed in step S10. When local data is available, a response is generated locally in step S11.

Step S12 determines whether additional target pharmaceutical suppliers remain to be processed. When additional target pharmaceutical suppliers remain, control returns to step S4 and the next target pharmaceutical supplier is processed. When no additional target pharmaceutical suppliers remain, the responses are formatted in step S13 for the prescriber and forwarded to the prescriber in step S14.

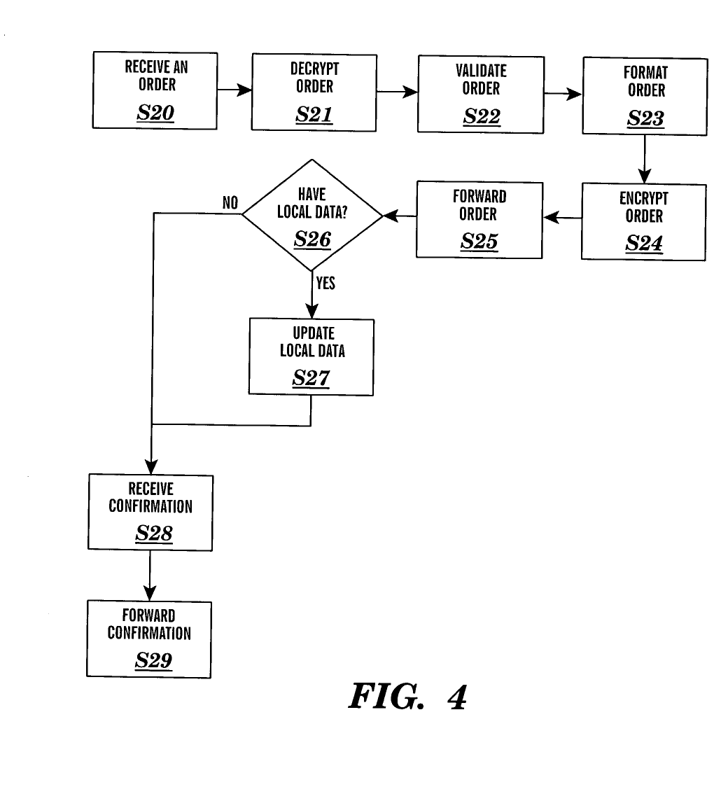

After reviewing the responses, a prescriber may decide to place an order. FIG. 4 depicts an exemplary method of processing an order according to one aspect of the invention. An order is received in step S20. For a patient's privacy, the order may be encrypted. In step S21, the order is decrypted and in step S22 it is validated. Once the order is decrypted and verified, it is formatted based on the selected pharmaceutical supplier in step S23. The reformatted order is then encrypted in step S24 and forwarded to the appropriate pharmaceutical supplier in step S25. In step S26, it is determined whether local data for the pharmaceutical supplier is available. When local data is present, it is updated in step S27. The pharmaceutical supplier can send a confirmation that the order was received and will be filled in step S28 and that can be forwarded to the prescriber in step S29. Alternatively, the local data can be updated after the confirmation is received.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of the invention as defined by the accompanying claims.

What is claimed is:

1. A method for electronically ordering a prescription, comprising:
receiving a prescription request from a prescriber;
processing the prescription request according to at least one selection criterion to identify at least one target pharmaceutical supplier from a group of pharmaceutical suppliers;
forwarding the prescription request to the at least one target pharmaceutical supplier;
receiving responses to the prescription request from the at least one target pharmaceutical supplier; and
forwarding the responses to the prescriber.

2. The method of claim 1, wherein the at least one selection criterion is selected from the group consisting of: a prescription price, a pharmaceutical supplier location, a prescription availability, an acceptance of an insurer, an availability of local data, an acceptance of a method of payment, a previous use, a delivery option, and an amount of time to fill.

3. The method of claim 1, further comprising:
validating the prescription request; and
formatting the prescription request for each of the at least one target pharmaceutical supplier prior to forwarding the prescription request.

4. The method of claim 1, further comprising:
generating a response for a target pharmaceutical supplier based on local data for the target pharmaceutical supplier; and
forwarding the generated response to the prescriber.

5. The method of claim 1, further comprising encrypting the prescription request prior to forwarding the prescription request.

6. The method of claim 1, further comprising:
receiving an order request from the prescriber; and
forwarding the order request to one of the at least one target pharmaceutical supplier.

7. The method of claim 6, further comprising:
validating the order request; and
formatting the order request for the one of the target pharmaceutical supplier prior to forwarding the order request.

8. The method of claim 6, further comprising encrypting the order request prior to forwarding the order request.

9. The method of claim 1, wherein the prescription request includes at least one selection criterion.

10. A method for electronically ordering a prescription, comprising:
receiving a prescription request from a prescriber;
processing the prescription request according to at least one selection criterion specified in the prescription request to identify target pharmaceutical suppliers from a group of pharmaceutical suppliers;
forwarding the prescription request to the target pharmaceutical suppliers;
receiving responses to the prescription request from the target pharmaceutical suppliers;
forwarding the responses to the prescriber;
receiving an order request from the prescriber for one of the target pharmaceutical suppliers;
validating the order request; and
forwarding the order request to the one of the target pharmaceutical suppliers.

11. The method of claim 10, further comprising:
generating a response for a target pharmaceutical supplier based on local data for the target pharmaceutical supplier; and
forwarding the generated response to the prescriber.

12. A system for electronically ordering a prescription, comprising:
a prescriber system for receiving a prescription request from a prescriber and for forwarding responses to the prescriber;
a processing system for processing the prescription request according to at least one selection criterion to identify at least one target pharmaceutical supplier from a group of pharmaceutical suppliers; and
a supplier system for forwarding the prescription request to the at least one target pharmaceutical supplier and for receiving responses to the prescription request from the at least one target pharmaceutical supplier.

13. The system of claim 12, further comprising:
a validation system for validating the prescription request; and
a formatting system for formatting the prescription request for each of the at least one target pharmaceutical supplier.

14. The system of claim 12, further comprising an encryption system for encrypting the prescription request.

15. The system of claim 12, wherein the prescriber system further receives an order request from a prescriber, and wherein the supplier system further forwards the order request to one of the at least one target pharmaceutical supplier.

16. A program product stored on a recordable medium for electronically ordering a prescription, which when executed, comprises:
program code for receiving a prescription request from a prescriber;
program code for processing the prescription request according to at least one selection criterion to identify at least one target pharmaceutical supplier from a group of pharmaceutical suppliers;
program code for forwarding the prescription request to the at least one target pharmaceutical supplier;
program code for receiving responses to the prescription request from the at least one target pharmaceutical supplier; and
program code for forwarding the responses to the prescriber.

17. The program product of claim 16, further comprising:
program code for validating the prescription request; and
program code for formatting the prescription request for each of the at least one target pharmaceutical supplier prior to forwarding the prescription request.

18. The program product of claim 16, further comprising program code for encrypting the prescription request prior to forwarding the prescription request.

19. The program product of claim 16, further comprising:
program code for receiving an order request from the prescriber; and
program code for forwarding the order request to one of the at least one target pharmaceutical supplier.

20. The program product of claim 19, further comprising:
program code for validating the order request;
program code for formatting the order request for the one of the at least one target pharmaceutical supplier prior to forwarding the order request; and
program code for encrypting the order request prior to forwarding the order request.
